(19) United States
(12) Patent Application Publication
(10) Pub. No.: US 2025/0177668 A1
(43) Pub. Date: Jun. 5, 2025

(54) Nasal Spray Device and Method of Nebulizing a Liquid Formulation

(71) Applicant: Medspray B.V., Enschede (NL)

(72) Inventors: Franciscus Martinus Verhoeven, Enschede (NL); Comelis Johannes Maria Van Riijn, Amsterdam (NL)

(21) Appl. No.: 18/845,046

(22) PCT Filed: Mar. 9, 2023
(86) PCT No.: PCT/IB2023/052265

§ 371 Date: Sep. 9, 2024

(30) Foreign Application Priority Data
Mar. 9, 2022 (NL) 2031214

(51) Int. Cl. A61M 15/08; A61K 9/00; A61M 11/00; A61M 15/00

(52) U.S. Cl. CPC A61M 15/08; A61K 9/0078; A61M 11/007; A61M 15/009; A61M 2205/0211

(57) ABSTRACT

A nasal spray device comprises a non-pressurized reservoir for containing a liquid formulation; a spray nozzle; and a manually energizable pump, configured to pressurize a quantity of said liquid formulation and causing the liquid formulation to move through the spray nozzle such that the liquid formulation is discharged from the spray nozzle as an aerosol spray. The spray nozzle comprises a membrane having a plurality of micropores of a substantially identical size below 8 micron and beyond 3 micron through which the liquid formulation passes as the liquid formulation is discharged from the device. Said membrane is configured to release said dose of said liquid formulation in a period of at least 500 milliseconds as a conically shaped plume having an apex. An energy of less than 20 Joule/gram is being dissipated in the liquid to preserve the molecular integrity of macromolecules within the liquid.

The present invention relates to a nasal spray device, comprising a nostril cap having an end portion to be received inside a nostril cavity of a user, wherein said nostril cap comprises a spray cavity that opens through said end portion, and wherein said nostril cap comprises a spray nozzle that is configured and suspended to receive a liquid formulation at a inlet and to release a liquid spray of said liquid formulation at an outlet that opens into said spray cavity.

Current manually operable nasal spray devices deliver a nasal spray that is generated by a pressurized swirl nozzle, also referred to as hollow cone nozzle. A stationary core inside such nozzle induces a rotary fluid motion which causes swirling of fluid in a swirl chamber. A liquid sheet film is discharged from the perimeter of an outlet orifice, producing a characteristic hollow cone spray pattern. Air or other surrounding gas is drawn inside the swirl chamber to form an air core within the swirling liquid. Many geometries of fluid inlets are used to produce this hollow cone pattern depending on the nozzle capacity and materials of construction. These swirl nozzles produce droplets with a more or less random size of between 20 and 200 micron within a relatively broad droplet size distribution having typically an average droplet size between 50 and 80 micron. A volume flow or discharge rate of sprays generated with swirl nozzles is relatively high and typically larger than 200 microlitre per second.

Another conventional technology for nebulization is based on a flexible mesh mounted on a piezoelectric lead zirconate titanate (PZT) ring actuator (ultrasound transducer) that stretches and vibrates, thereby expelling droplets with relatively random sizes typically in a range of between 1 and 10 micron. The vibrations extend to the liquid formulation and generate heat that is dissipated in the liquid, increasing the temperature of the formulation during the nebulization process.

Finally atomizing of a liquid formulation may be effected by crossing jets that are allowed to collide at a high velocity. This colliding jet (also called impinging jet) method is based on such collision at a large velocity of two jets, forming a thin liquid sheet at the location of impact (also known as a Savart sheet), that disintegrates into randomly sized droplets, typically with a broad size range of between 1 and 10 micron. To ensure that the two jets have sufficient velocity to form droplets smaller than 10 μm after rupture of the Savart sheet, the required system pressure is typical between 150-200 bar.

All these known nebulizing techniques require a substantial minimal energy to be supplied to the liquid and, hence, lead to a considerable impact of the liquid spray in the nose. The latter is not only a drawback from a sensory point of view to the user, but also leads to a relatively short dosage time. Typically a dose of 40 microlitre is being delivered in a dosage time of less than 200 milliseconds with such a conventional nasal spray device and most of the liquid spray happens to land no further than in the nasal vestibule. Especially many spray pump swirl nozzle devices deposit a significant amount of their drug in the anterior vestibule which is considered a less active region of the nasal cavity. This anterior vestibule deposition may be due to inertial impaction since most spray pumps are designed to release a large proportion of aerosol particles greater than 20 micron which exit the device in short time at a relatively high speed when actuated.

Moreover, the same high energy level and impact on the liquid associated with these known nebulizing techniques appears to destroy the integrity of delicate macromolecules, if residing in the liquid formulation. This renders known nebulizing devices and methods less suitable for nebulizing large macro molecules, like lipid particles that are often found in biological therapeutic formulations.

It is inter alia an object of the present invention to provide a nasal spray device that meets the above drawbacks without sacrificing substantial convenience to the user. In one aspect of the invention, it is an object to provide a nasal spray device that provides an improved coverage of the nasal cavity, particularly including the olfactory region of the nasal cavity. More particularly, in a still further aspect the invention has for its object to provide a nasal spray device that is capable of aerosolizing nanoparticles, that may comprise relatively long molecules, particularly fragile biological molecules, while substantially preserving their nanoparticle structure and molecular integrity.

In order to achieve the above object a nasal spray device of the type described in the opening paragraph, according to a first aspect of the invention is characterized in that that said spray nozzle comprises a membrane between said inlet and said outlet having a plurality of micropores of a substantially identical size of between 1 micron and 10 micron, particularly between 3 micron and 8 micron, that carry said liquid formulation to be discharged as a spray jet from the device, in that said micropores are configured to release a jet of microdroplets under an angle of deflection with respect to a axial centreline of said respective micropore and in that said micropores are distributed to form together a substantially conically shaped diverging spray plume having an apex.

The device according to the invention is based on a relatively tender Rayleigh breakup of the liquid formulation after having passed through relative short micropores of a defines size. This will create a soft mist of ultra-fine droplet having substantially identical size, independent of their propagation velocity. Particularly arranging and configuring the micropores such that they will create a slowly propagating substantially conically diverging plume contributes to an improved nasal coverage of the nostril region, notably in a direction travers to the direction of propagation. The deflection of the individual spray jets that are released by the micropores may particularly be effected and configured by means of the technique as disclosed in co-pending European patent application EP . . . by applicant, the content of which is herewith incorporated by reference.

The invention is thereby based on the recognition that Rayleigh breakup of the liquid formulation by means of micropores that are smaller than 8 micron will deliver spray droplets of a substantially identical initial size of typically between twice and three times the size of the micropores. These still relatively small, micron sized droplets together form an ultra-fine spray plume that will be retarded substantially instantaneously once released in and exposed to ambient air due to the relatively small kinetic energy of the individual droplets. Particularly the resulting spray plume will be slowed down to have a propagation velocity not exceeding the order of 1 m/s. It has been found that the turbulence within this spray plume together with such slow rate of movement delivers an unprecedented coverage over basically the entire nasal cavity, including the higher olfactory region.

It has surprisingly be found that particularly the inferior and middle turbinate may be targeted by a first particular embodiment of the nasal spray device according to the invention that is characterized in that that said conically shaped spray plume has an apex of around 5 degrees.

A second particular embodiment of the nasal spray device, on the other hand, is characterized in that that said conically shaped spray plume has an apex of between 5 and 30 degrees, particularly of between 20 and 25 degrees, and more particularly of around 20 degrees. This spray device may advantageously be used to target particularly the nostril and/or olfactory region and nasopharynx of the nasal cavity.

A further embodiment of the nasal spray device according to the invention is characterized in that a chamber is provided for containing a predetermined dose of said liquid formulation, particularly having a volume of between 25 and 150 microlitre, in that a manually energizable positive displacement pump is configured to pressurize said dose of said liquid formulation and forcing the liquid formulation under an elevated operating pressure of between 5 and 15 bar to said inlet of said spray nozzle that releases said predetermined dose of said liquid formulation.

The present invention particularly provides a nasal spray device that generates a so-called micro-jet spray. A micro-jet spray consists of a number of concurrently emitting jets, in which each jet will initially breakup into a mono-disperse primary droplet train according to a jet breakup mechanism. As a result, consecutive primary droplets have substantially identical size and propagate downstream of the nozzle in a same direction.

Typically a diameter of a primary droplets is between twice and three times the diameter of the micropore that released it. This means that according the invention the primary droplets will have an identical size of between 6 and 30 micron, depending the size of the micropores by which they are generated. Eventually individual droplets may merge due to mutual coalescence which will cause a shift and certain spread in the droplet size distribution throughout the spray. The droplet size distribution remains nonetheless relatively sharp and well defined.

At this instance it is noted that the size of a micropore is being defined in the present application as representing the diameter of a circle having a same surface area as a cross sectional surface area of said micropore. Likewise the size of a droplet is being defined as the diameter of a sphere having a same volume as the volume contained by said droplet. Further the expressions nebulizer, atomizer and spray device are used interchangeably without entailing any essential difference, unless explicitly stated otherwise.

The nasal spray device according to the invention operates on basis of a pump, particularly one that is manually energizable, to deliver an ultra-fine mist of these relatively small droplets. Due to their relatively low momentum, these droplets tend to slow down relatively rapidly as the encounter ambient air. In practice a residence time of the order of 500 milliseconds and even longer has been observed. This slow release of relatively small droplets offers a slowly moving soft mist of said liquid formulation.

A patient may inhale gently via the nostril which will convey the soft mist droplets to nasal target areas further downstream the nasal cavity. A majority of the uniformly sized droplets will particularly be capable of targeting the respiratory and olfactory nasal region. This enhanced penetration is attributed to the fact that the spray device of the invention is designed to generate small, and also slowly moving particles that traverse the nasal cavity at a normal inhalation rate, thereby minimizing inertial impaction anterior to the nasal valve.

Due to the relatively sharp droplet size profile of the spray, as is associated with Rayleigh formation, only a small fraction of the soft mist spray, if any, consists of droplets of too small size, particularly smaller than 10 micron, such that they may escape further into the respiratory system of the user, particularly into the lungs.

Targeting the olfactory region is especially aimed at for topical drugs that can be up taken by the brain directly, thus avoiding a blood-brain barrier. Also a clearance of nasal fluid in the olfactory region is considerably lower than in the vestibule region, allowing the formulation a longer time to be up taken by the nasal mucus tissue. These factors greatly contribute to a significantly enhanced uptake efficiency and nasal coverage. A pump based nasal spray device according to the invention comprises a nozzle that generates a slowly moving fine mist of the liquid formulation that may be able to reach and cover up to more than 80% of the internasal mucus layers.

In a further aspect, the present invention aims at providing a method that is suitable for nebulizing a liquid formulation that preserves the molecular integrity of any macromolecules residing within said liquid, at least to a large extent. To that end, a method for nebulizing a liquid formulation, particularly a liquid formulation containing macro molecules, more particularly containing biological molecules, in which said liquid formulation is subjected to a treatment to convert said liquid formulation into a mist of said liquid formulation, is according to the invention characterized in that said liquid formulation is subjected to an energy not exceeding 20 Joule/gram, preferably not exceeding 10 Joule/gram, while converting said liquid formulation into a mist of said liquid formulation, particularly forming a soft mist spray plume.

It has been found that if only a limited amount of energy is dissipated during atomizing of the liquid, at least a majority of fragile macromolecules, like complex proteins, peptides, long chain DNA & RNA, large vesicles, liposomes and antibodies, will survive the atomizing treatment. This may be achieved by means of a process Rayleigh breakup to create a fine mist of said liquid formulation. Accordingly, a preferred embodiment of the method according to the invention is characterized in that said liquid formulation is pressurized to an operating pressure to create at least a pressurized dose of said liquid formulation, and in that said pressurized dose of said liquid formulation is forced through at least one micropore, particularly trough a plurality of micropores, in a membrane of a Rayleigh break-up type spray nozzle.

The invention particularly relates to a nasal spray device that may be used to aerosolize a pharmaceutical formulation for nasal administering. A specific embodiment of the nasal spray device according to the invention, to that end, is characterized in that said reservoir contains a pharmaceutically active liquid formulation, particularly one containing nanoparticles, such as formulations containing complex proteins, peptides, long chain DNA & RNA, large vesicles, liposomes and antibodies.

As an example, the pump based nasal spray device of the invention will be able to protect a user against allergic attacks or diseases caused by microorganisms or viruses, such as the SARS-Cov-2 viruses, with an appropriate anti-allergenic or prophylactic formulation. Current prophylactic formulations against SARS-Cov-2 viruses to prevent that the virus will bind to epithelial cells in the nasal cavity are, angiotensin receptor blockers, ACE inhibitors, heparin and enoxaparin derivatives. Other prophylactic medicines and vaccines include ivermectin, antibodies, glycoproteins, mRNA lipid nanoparticles, and other biologically active agents. The pump based nasal spray device of the invention may comprise a nozzle that preserves the integrity of such formulation, especially in the case of biological formulations, that are generally based on large molecules and/or nanoparticles, such as formulations containing complex proteins, peptides, long chain DNA & RNA, large vesicles, liposomes and antibodies. Current fine mist nebulizers for nasal use, on the other hand, have been found to destroy the molecular integrity of large molecules that are present in many mRNA lipid nanoparticle vaccines. This renders these conventional nebulizers unsuitable for nasal administration of many protein based or other biological drugs.

The nasal spray device according to the invention may be configured to target the deeper respiratory system, particularly the lungs, as well besides the nasal cavity. To that end a specific embodiment of the nasal device according to the invention is characterized in that said membrane of said spray nozzle comprises a second group of micropores of substantially identical size below 3 micron through which the liquid formulation passes as the liquid formulation is discharged from the device. These relatively small micropores will deliver correspondingly smaller droplets, typically smaller than 10 micron, that will be able to pass the nasal cavity to eventually target the deeper tracheal and/or even bronchial areas of the respiratory system.

In a preferred embodiment the nasal spray device according to the invention is characterized in that said pump comprises a manually actuated piston that pressurizes said dose of said liquid formulation to said operating pressure in a single stroke of said piston. A piston that pressurizes the entire dose in a single stroke provides convenience to the user, particularly if the pump mechanism is further configured to automatically fill said dose in a dedicated predetermined and defined volume of the device prior to pressuring it. Said pump may comprises a multi shot nasal pump that is provided with a dosing chamber for holding said dose of said liquid formulation at least temporarily and releasing said dose to said spray nozzle under said operating pressure. Alternatively said pump may be formed by a medical syringe.

A specific embodiment of the nasal spray according to the invention is characterized in that said pump opens into a male Luer tip, in that the nostril cap is receivable onto said male Luer tip having an inlet that provides a female Luer slip connection with said male Luer tip and having an outlet to release said nasal spray, and wherein said nostril cap comprises said spray nozzle between said inlet and said outlet. This embodiment particularly allows a standard medical syringe to be used as the pump and reservoir for containing the liquid formulation. Simply mounting the nostril-cap, embodying the spray nozzle according to the invention, on top of the syringe transforms the syringe into a nasal spray device that outperforms many conventional nasal spray devices. Both the syringe and the nostril-cap may be provided as low cost disposable articles.

A further specific embodiment of the spray device according to the invention is characterized in that said nostril-cap comprises at least one inhalation channel that carries a parallel airflow of ambient air along with said spray towards the outlet, while the user inhales. Such an inhalation channel may for instance be provided in a wall of said nostril-cap, opening into environment. This induces additional airflow through the nostril-cap consisting of ambient air, while the user inhales. The spray droplets may piggyback on this additional airflow or may be carried into its slipstream to be delivered deeper into the nasal cavities. Even remotely located nasal areas inside the nasal cavity may be targeted this way. A preferred embodiment of the nasal spray device is characterized in that said nostril-cap tapers down towards said outlet to have a close nasal fit.

In a preferred embodiment said membrane comprises a ceramic layer, wherein said micropores extend over a thickness of said ceramic layer. The micropores may for instance be formed by etching and/or using micro-machining using photolithography as customary in nowadays semiconductor technology. In that respect, a specific embodiment said ceramic layer is a silicon nitride layer lying on a carrier body of a semiconductor material, particularly of silicon, wherein said carrier body is provided with at least one cavity underneath said nitride layer, wherein said at least one cavity opens downstream into at least one micropore of said plurality of micropores, and wherein said at least one cavity is connected upstream to a outlet of said pump.

The present invention further relates to a method of spraying a liquid formulation, wherein said liquid formulation is pressurized to an operating pressure of between 5 and 15 bar using a pump to create at least a pressurized dose of said liquid formulation, wherein said pressurized dose of said liquid formulation is forced through a group of micropores in a membrane of a Rayleigh type spray nozzle, said micropores having a size smaller than 8 micron, to cause said liquid formulation to breakup in at least one spray plume of liquid droplets having substantially an identical initial droplet size, and wherein said spray plume is caused to propagate with a plume propagation velocity of less than 1 m/s, particularly in a nasal cavity of a user. Such spray plume appears particularly appropriate and effective as a nasal spray for nasal administration of said liquid formulation.

The present invention particularly relates to a nasal spray device and method for delivering a spray from a liquid formulation comprising nano-particles that are capable of preserving the integrity of said nano-particles while said liquid formulation is converted into a spray. To that end, a special embodiment of the method of spraying a liquid formulation is characterized in that said liquid formulation contains nano-particles of a size δ and said liquid droplets contain at least one nano-particle of said nano-particles, wherein said nano-particles have a maximum size δ max before rupture upon elongation; wherein said liquid formulation is subjected to a shear rate γ [per second] while passing through a micropore; and wherein said liquid formulation is exposed within said micropore to said shear rate γ during a shear time Δt that is less than δ max/(δ·γ) seconds.

In a further embodiment the nasal spray device and method according to the invention are characterized in that said liquid formulation comprises nano-particles of a size δ and said liquid droplets contain said nano-particles, wherein said nano-particles have a maximum size δmax before rupture upon elongation; and wherein said micropores have a length L that is smaller than the pore diameter D times δmax/δ (L<D·δ max/δ). In case of tapering pores the size or diameter D of the micropore is then taken as the largest diameter of the micropore. For long chain molecules, such as mRNA, etc, the length of the micropore L should be less than the total length A of the long chain molecule (L<λ), in particular L is less than 1 micron.

The nasal spray device and method according to the invention are particularly suitable for spraying a liquid formulation that comprises nano-particles taken from a group, containing complex proteins, large biological molecules, long chain DNA & RNA, large vesicles, liposomes, bacteriophages and antibodies, while preserving the integrity of such nano-particles.

Hereinafter, the invention will be described in further detail with reference to a specific embodiment and an accompanying drawing. In the drawing:

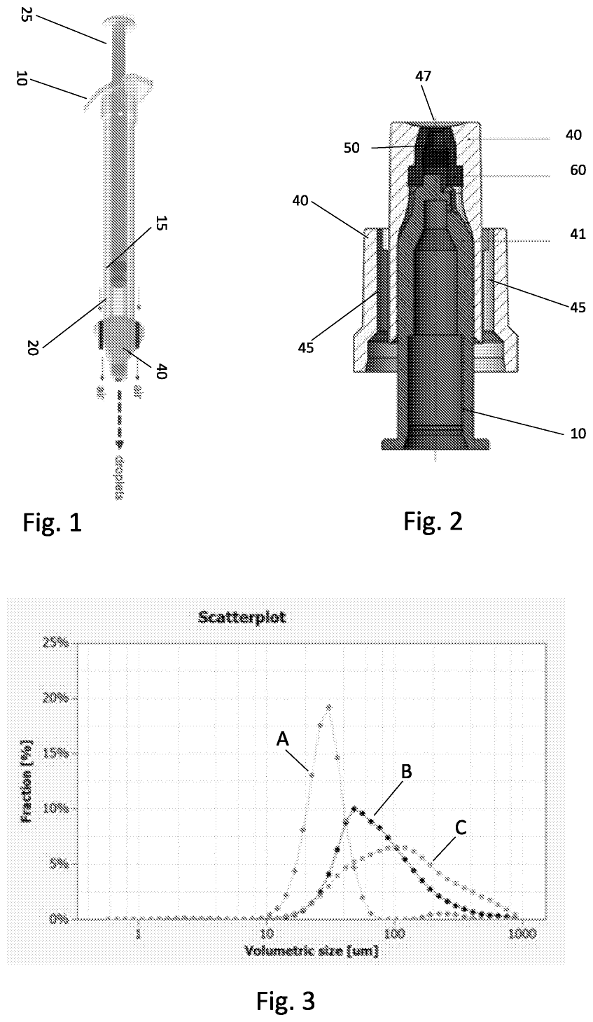

FIG. 1 shows a specific example of a nasal spray device according to the invention;

FIG. 2 shows a schematic drawing of part of the device of FIG. 1;

FIG. 3 provides a comparison of a droplet size distribution between the device of FIG. 1 and a conventional swirl nozzle nasal spray device;

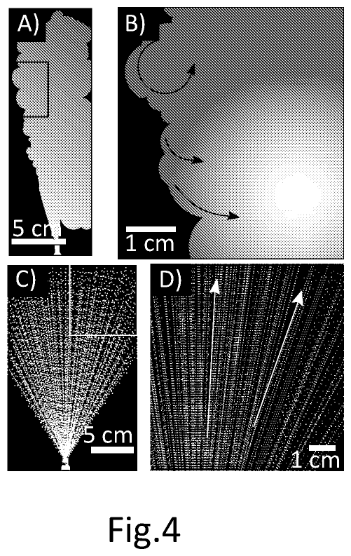

FIG. 4a-d shows high-speed photography capture images of a spray generated by a nasal spray device according to the invention as compared to that of a conventional swirl nozzle nasal spray device;

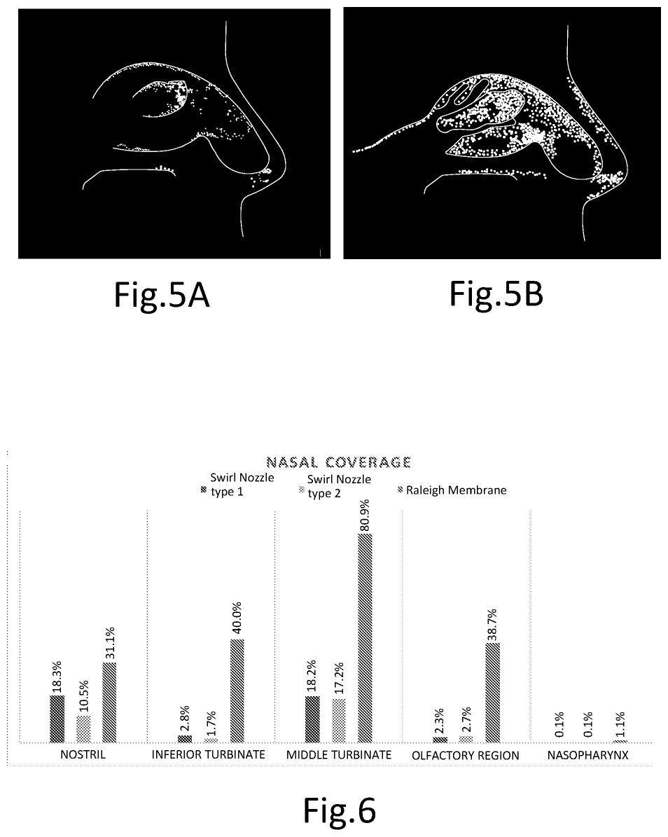

FIG. 5A shows a high speed camera image of a nasal coverage of a liquid spray by means of said conventional swirl nozzle nasal spray device;

FIG. 5B shows a high speed camera image of a nasal coverage of a liquid spray by means of said nasal spray device according to the invention;

FIG. 6 shows a comparison of a total nasal coverage between different types of spray devices;

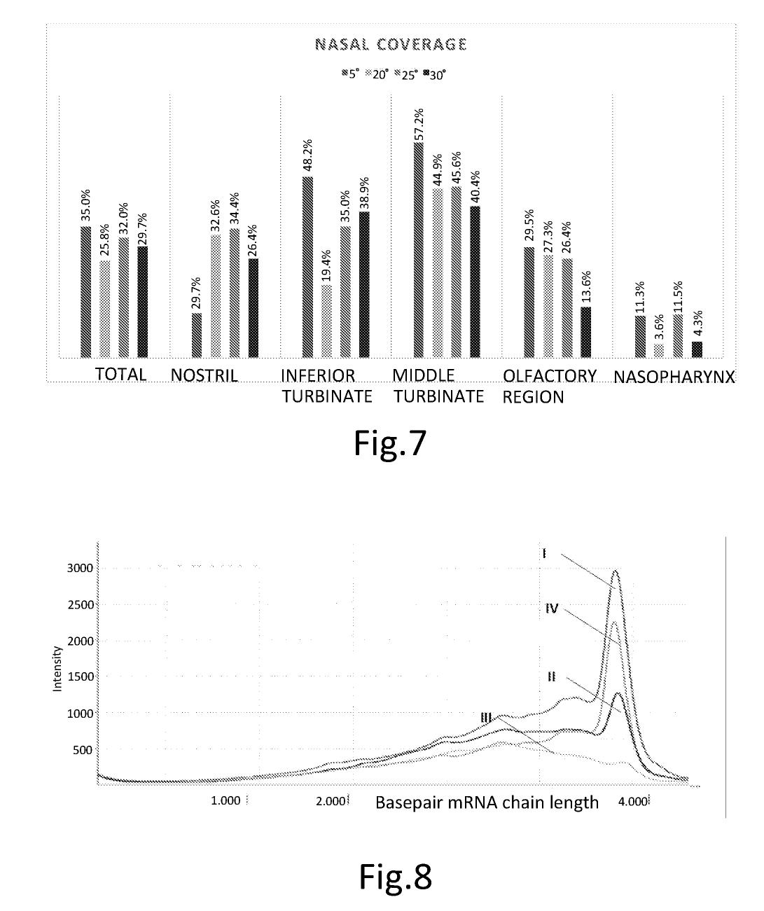

FIG. 7 shows an affect of the apex of spray plume on the target location by a spray plume that is generated by a spray device according to the invention;

FIG. 8 provides a graphical representation of the performance of a spray device according to the invention for spraying a liquid formulation containing mRNA molecules as compared to conventional spray devices;

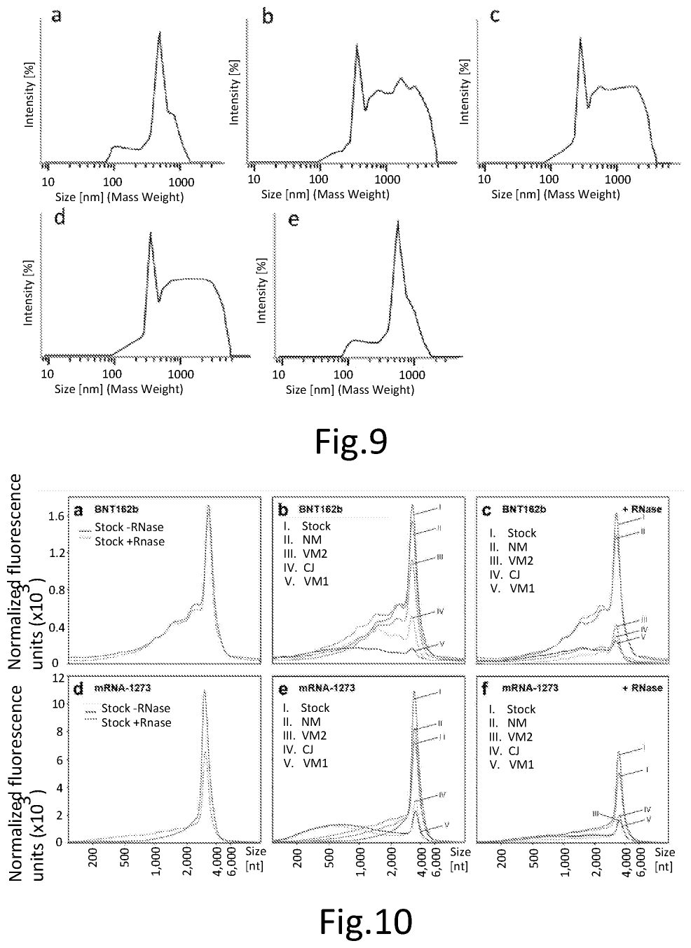

FIG. 9a-e show the particle size distributions of mRNA-1273 after nebulization, as obtained from Dynamic Light Scattering (DLS) in mass weight mode, comparing:
a. the initial stock formulation;
b. after nebulization with vibrating mesh VM1 at 35 J/g;
c. after nebulization with vibrating mesh VM2 at 18 J/g;
d. after nebulization with colliding jet (CJ) at 22 J/g; and
e. After nebulization with nanotech membrane (NM) method according to the invention at 2 J/g.

FIG. 10a-f show electropherograms for the BNT162b (a,b,c) and mRNA-1273 (d,e,f) vaccines before and after nebulization with vibrating mesh type 1 (VM1; blue), colliding jet (CJ; yellow), vibrating mesh type 2 (VM2; red), and nanotech membrane (NM; green), all compared to the stock vaccines with and without RNase to demonstrate the effect of nebulization on mRNA integrity;

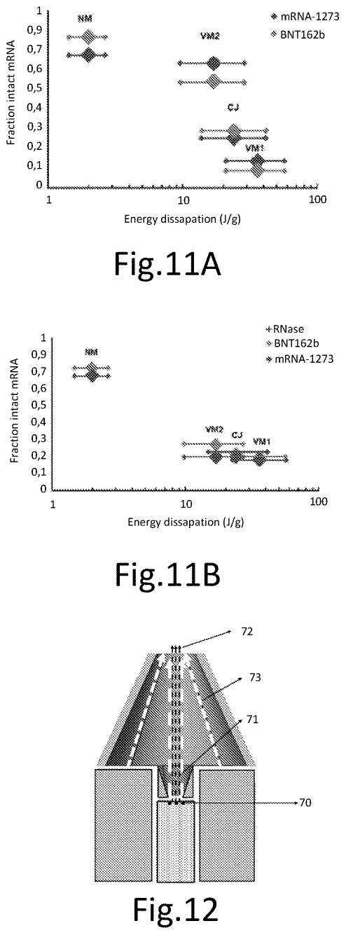

FIG. 11A shows the fraction of intact mRNA versus nebulization energy dissipation without adding RNase;

FIG. 11B shows the fraction of intact mRNA versus nebulization energy dissipation with RNase added after nebulization; and

FIG. 12 shows a schematic representation of a particular embodiment of a nasal adapter for use with nasal spray device according to the invention.

It is noted that the figures are drawn purely schematically and not necessarily to a same scale. In particular, certain dimensions may have been exaggerated to a more or lesser extent to aid the clarity of any features. Similar parts are generally indicated by a same reference numeral throughout the figures.

As an example of a nasal spray device according to the invention, FIG. 1 shows a standard medical syringe 10 that has a movable piston 15 that is manually operable by means of a stem 20. In practice it is possible to exert an operating pressure of between 2 and 10 bar of a liquid that is contained in the reservoir 25 contained in the syringe 10 and to force said liquid at such operating pressure into the nostril-cap 40 that is mounted by means of a male-female slip connection on a tip of the syringe and that contains a spray nozzle 50 to receives said pressurized liquid.

The nostril-cap is shown in more detail in FIG. 2 and basically comprises a plastic cone that is fitted on the tip of the syringe 10. The nostril cap 40 is received sealingly onto the male Luer tip of the syringe 10 by means of an internal female Luer slip connection at an inlet of said nostril-cap 40. The nostril-cap comprises said spray nozzle 50 inside an internal cavity proximal to an outlet 47 side. The nozzle 50 is configured and connected for receiving said liquid at said operating pressure and converting it into a ultra-fine soft mist. To that end the nozzle 50 comprises a spray microchip of silicon that is fitted sealingly inside an internal cavity of a plastic adaptor body 60. Said adaptor body 60 facilitates easy handling and assembly of the nozzle device and moreover may accommodate one or more filters upstream of the spray chip 50.

The spray chip 50 holds a brittle ceramic silicon nitride membrane of typical 1 micron thickness. This membrane comprises a plurality of micropores extending through said thickness. A thin silicon oxide layer may be provided between the nitride layer and the silicon body. In such case, the micropores will extend through this oxide layer as well. As a result the micropores are in open communication with one of a number of subjacent cavities that are etched through said silicon body from a backside to its front surface. These cavities convey the pressurized liquid to the micropores. The micropores were created with high precision to have a substantially identical size of 4 micron in diameter. To that end, like the other structures of the micro-chip, the micropores are preferably created by photo lithographic aided etching and deposition techniques as customary in modern semiconductor manufacturing technology. Using these techniques at least a portion of the micropores is modified and configured to release their microjet under a predefined diverging angle of deflection with respect to their centreline. These micropores are distributed and aligned over said membrane surface such that together they will create a substantially cone shaped spray pattern, said cone having a predefined apex of between 5 and 30 degrees, typically between 10 and 25 degrees. The liquid is forced through said micropores, during operation, and breaks up into small droplets as the cohesion is no longer capable of concurring with the surface tension. This physical phenomenon is generally referred to as Rayleigh breakup and cause the liquid to breakup is essentially equal droplets of a size that is initially approximately between twice and three times the size of the releasing micropore. Eventually individual droplets may merge due to coalescence which will cause a shift and certain spread in the droplet size distribution throughout the spray. FIG. 3 provides a plot in which curve A represents a typical droplet size distribution of the spray generated by this nozzle. The droplets have grown to between 20 and 40 micron. This spray nonetheless maintains a relative sharp symmetrical profile as compared to a spray that is generated by means of for instance a swirl nozzle. In order to demonstrate this, FIG. 3 also contains two comparison curves B, C that represent a typical profile of the droplet size distribution of a spray that was generated using a conventional swirl nozzle.

To enhance the penetration and total coverage into the nasal cavities by a nasal spray generated by the depicted spray device, the nostril-cap 40 comprises a number of inhalation channels 45 aside from the central cavity that carry a parallel airflow of ambient air along with said spray towards the outlet 47, while the user inhales. The spray chip of the nozzle 50 is configured to release a slowly moving liquid spray. Typically a dose of the order of between 25 and 150 micro litre is released in a relatively long period of at least 500 milliseconds by said nozzle 50.

A number of studies have proven that such a slowly moving nasal spray is an excellent method of delivering topical medication beyond the nasal valve region. This enhanced delivery and penetration is attributed to the fact that small, slow moving liquid particles are able to traverse the nasal cavity at a resting breathing rate, thereby minimizing inertial impaction anterior to the nasal valve. In addition to the delivery device, a combination of other factors also contributes to the efficacy of intranasal formulations. These include drug formulation characteristics, site of deposition and patient friendly use of the delivery device. These excellent deposition characteristics are achieved with a relatively simple spray pump provided by a medical syringe 10.

To explain the striking difference in droplet deposition for a soft mist nozzle of the nasal spray device according to the invention as compared to a standard swirl nozzle, a high-speed photography capture of the initial droplet velocity and plume formation (FIG. 4A-4D). These images show spray droplet trajectories for the two spray nozzles. FIG. 4A shows the spray pattern of a spray generated by means of a soft mist nozzle according to the invention, while FIG. 4B is a magnification of the rectangular area that is marked area in FIG. 4A. Eddies can be observed as indicated by the arrows. FIG. 4C shows the spray pattern of a spray generated by means of a standard swirl nozzle, while FIG. 4D is a magnification of the rectangular area that is marked area in FIG. 4C. Straight droplet trajectories can be observed as indicated by the arrows in FIG. 4D.

The spray plume of the swirl nozzle has an initial velocity of about 13 m/s, spraying a total volume of 100 μL in approximately 100 ms. The spray plume by the nozzle of the present invention, however, has a considerably lower initial velocity of less than 1 m/s, particularly about 0.7 m/s, and sprays 45 μL in no less than 500 ms. Furthermore, the soft mist nozzle according to the invention produces relatively small droplets (Dv50 20-25 μm) as compared to a standard nasal swirl nozzle (Dv50 60 μm) as indicated by the comparative plots of FIG. 3. Essentially the standard swirl nozzle produces a short burst of relatively heavy droplets that travel all in straight trajectories in an upwards direction, as shown in FIGS. 4C and 4D. The soft mist nozzle of the nasal spray device according to the invention, on the other hand, produces droplets in a turbulent droplet cloud travelling relatively slowly in all directions, as shown in FIGS. 4A and 4B. The ballistic trajectories of the droplets of the swirl nozzles causes most droplets of the spray to be deposited only at the entrance of the nasal cavity. This is an inherent property of swirl nozzles, where the medium droplet size is determined by a competition between the surface tension of the liquid and inertia. In this case the medium drop size scale proportional to v^{2/3}, with v the velocity. This implies that relatively small droplets, as desired for deeper nasal administration, may only be obtained by increasing the velocity within a swirl nozzle.

The droplets size of the soft mist nozzle of the nasal spray device according to the invention, on the other hand, is predominantly determined by the size of the micropores, independent of the spray velocity. First of all this allows a better control of the droplet size distribution within the spray as the micropores may be created with great precision and definition. But, at least equally well important, this allows downscaling of the droplet size without increasing the droplet velocity to render an ultra fine slowly moving nasal spray that gives an unprecedented intranasal coverage in a handheld, manually energizable pump driven device.

FIGS. 5A and 5B show the result of an experimental setup to demonstrate the unexpected nasal coverage by the nasal spray device according to the invention. The setup uses a commercially available transparent nasal cast by Koken Co. Japan to mimic the human nasal region with an intranasal volume of 38,6 cm³ in combination with a fluorescent liquid formulation under blacklight conditions as recorded by a standard camera. In both cases the angle of insertion is about 45 degrees at an insertion depth of 1 cm. The liquid formulation that is being used contains 1 mg/ml calcein dissolved in a 25% glycerol-water mixture to improve the optical visibility of the evolving spray. The administered dose has a volume of 100 microlitre.

FIG. 5A shows the proliferation of a nasal spray within the nasal cavities using a standard conventional pump driven nasal spray device with a swirl nozzle. The figure shows large droplets and concentrations at the entrance of the nasal cavity with only a poor overall coverage of the nasal mucosa. Only 18% of cavity is covered with standard nasal spray device, resulting mainly in big droplets deposition.

FIG. 5B, on the other hand, shows the proliferation and spread of the same liquid formulation over the nasal cavity using a nasal spray device according to the invention. The high speed camera image shows a slowly moving aerosol cloud that travels and deposits uniformly over the cast model. In the end ultra-fine droplets are highly distributed over substantially the entire nasal cavity, including the higher olfactory region. This soft mist nasal spray covers 58% of nasal mucosa with fine mist of small micron sized droplets.

A spray device according to the invention comprising a group of 48 micropores having identical size of 4 micron and creating a soft mist plume with a cone apex of approximately 20 degrees has been setup and tested. This device has been compared for nasal delivery to a standard commercially available swirl nozzle by Aeropump and a state of the art MAD300 swirl nozzle by Teleflex. Particularly a coverage of different regions within the nasal cavity has been investigated with anyone of these spray devices. FIG. 6 shows a graphical representation of the test results that show an overwhelming coverage in every region by the device according to the invention, denoted as “Rayleigh Membrane” in the figure. Particularly the spray device according to the invention delivered over 80% coverage of the middle but also out-performed the other devices by more than a factor in the other areas.

The effect of the apex of the cone spray created by the same device according to the invention was further investigated. This is shown in the graphical representation of FIG. 7. Clearly an apex of around 5 degrees seems favourable to target particularly the inferior and middle turbine, while an apex of between 20 and 25 degrees is typically advantageous, and preferably around 20 degrees, if the olfactory region and nasopharynx are to be targeted. Also the droplet size range within the spray influences where the liquid formulation will be deposited after inhalation. Droplets in the 0.5-4 μm range will be mainly deposited in the alveolar regions, droplets of 4-10 μm in the central and peripheral airways, and droplets larger than 10 μm mainly in the oral cavity, nose or throat.

The ability to produce an ultra-fine mist of relatively small droplets with an unprecedented nasal coverage renders the spray device according to the invention particularly suitable for nasal administration of pharmaceutically active liquid formulations. This particularly relates to biological drugs, like prophylactic medicines and vaccines that may include antibodies, glyco-proteins, mRNA containing lipid nano-particles, and other biologically active agents. These generally delicate molecules or molecular nano-particle structures, however, tend to break or otherwise deteriorate when exposed to extreme shear forces that are exerted on them when using a conventional swirl nozzle or a vibrating mesh for nebulizing the liquid formulation.

This is demonstrated in FIG. 8 showing a plot of the molecule size of mRNA in a liquid formulation before and after aerosolizing with different nebulizers of COVID-19 vaccine BNT162b from Pfizer/Biontech. The vaccine formulation contains lipid nano-particles with an average size δ of 300 nm, which nanoparticles further comprise mRNA chains of ca 4.000 basepairs encoding for the virus spike protein with a chain length λ of approximately 4.000×0.34 nm (=1.36 micron). A reference curve I gives the molecule length of mRNA in the Pfizer/Biontech stock liquid, while curves II and III depict the molecule length after the same liquid formulation was sprayed using commercially available vibrating mesh nebulizers by Aeroneb and Pari eFlow respectively. Clearly the average mRNA molecule length diminished due to spraying with these current spray devices. This renders these conventional devices unsuitable for spraying of relatively fragile molecular nanostructures and long molecules.

Nebulization of large macro molecules, particularly biological molecules like those found in mRNA therapeutics, however, could otherwise favourably be used to directly target the respiratory tract. A promising prospect is that mucosal administration of lipid nanoparticle (lipid nano particle)-based mRNA vaccines may lead to a more efficient protection against respiratory viruses. However, a conventional nebulization process will rupture the lipid nano particle vehicles and degrade the mRNA molecules inside. The present invention, however, provides a novel nebulization method that proves to be able to preserve substantially the integrity of vaccines, as tested with two SARS-CoV-2 mRNA vaccines. Particularly a lower energy level in generating lipid nano particle droplets using the new nebulization method contributes to safeguard the integrity of the lipid nano particle and vaccine. Table 1 shows a comparison of nebulization techniques and their different energy dissipation levels

TABLE 1

| Technique | Energy input [J/g] |
|---|---|
| Vibrating mesh, VM | 35 |
| Colliding jet, CJ | 22 |
| Rayleigh nanotech membrane, NM | 2 |

10 The inventors have surprisingly found that lipid nano particles and mRNAs can be kept largely intact if the energy dissipation for nebulizing of the liquid formulation remains below a threshold value of 20 Joule/gram, for lipid nano particle integrity 5-10 J/g and for mRNA integrity 10-20 J/g for both vaccines.

The nasal spray device according to the invention, based on relatively tender Rayleigh breakup of the mRNA containing liquid formulation after having passed through relative short micropores of about 1 micron length, happens to preserve the mRNA molecule to a large extent, as indicated by curve IV in FIG. 8. Also the lipid nano-particle structure with an average size of δ=300 nm is preserved using the device according to the invention, contrary to the conventional vibrating mesh nebulizers. These vibrating mesh nebulizers cause a lot of fluid mechanical agitation in the formulation. In a device according to the invention the micropores have a rather short pore length L, such that the passage time Δt of the nanoparticles experiencing a (high) shear rate γ is short enough to prevent rupture of the lipid nano-particle structure. Further experiments show that for nano-particles with an average spherical diameter 6 with a maximum elongation upto δmax (before rupture occurs) can be preserved when the maximum shear time Δt is less than δmax/(δ·γ) seconds with δmax/δ is a number between 3 to 5.

This implies that the length L of the micropore preferably does not exceed the diameter D of the micropore, because the amount of shear on the nano-particle is proportional to the length L of the micropore and is inversely proportional to the diameter D of the micropore rate in a channel with diameter D.

A further finding behind the present invention is that for long chain molecules, such as mRNA with a chain length λ, the length of a micropore L, according to the invention, is preferably shorter than the total chain length λ of the long chain molecules. The breakage force on the long chain molecules, particularly mRNA, due to the presence in a shear field appears proportional to the chain length λ, when the molecule is fully stretched in the shear field. However when the mRNA molecule passes through a pore with a length L that is less than the chain length λ of the mRNA molecule, it is avoided that the molecule is entirely stretched. Instead it will partly maintain its initial globular shape, reducing the imposed stretching and ultimately breakage force on the molecule. The length of the micropore L according to the invention, hence, preferably is shorter than the total chain length λ of the long chain (mRNA) molecule.

In particular micropores with a length L less than 1 micron have been successfully tested for this purpose. This is shown by the curve IV in FIG. 8 that demonstrates the mRNA molecule length λ of the liquid formulation of the same Pfizer/Biontech mRNA liquid vaccine after the formulation was sprayed using the nasal spray device according to the invention having micropores of a length L of 1 micron that is smaller than the chain length λ of the mRNA molecule. This opens an entirely new opportunity of nasal administering bio-technological pharmaceuticals that are often based on biologicals and nanoparticles, such as formulations containing complex proteins, peptides, long chain DNA & RNA, large vesicles, liposomes, antibodies, and other biological compounds as their active ingredient.

FIG. 9a shows that the lipid nano particles containing mRNA-1273 vaccine before nebulization have a size range of 300-500 nm. Nebulization by vibrating mesh and colliding jet methods causes an overall shift to larger lipid nano particle diameters (cf. FIG. 9b-9d), indicating aggregation of lipid nano particles, whereas nebulization by the method according to the invention leaves the lipid nano particle size distribution largely unchanged (cf. FIG. 9e). To investigate whether the larger lipid nano particles are formed by aggregation of smaller or ruptured lipid nano particles during nebulization, experiments were performed with a specific anti-aggregation formulation. Notably, a broad range of small to large lipid nano particles was detected for the vibrating mesh and colliding jet samples, indicating that the aggregates above 1 μm observed after nebulization can reasonably be assumed to be due to aggregation of smaller and ruptured lipid nano particles.

To assess the effect of the different nebulization methods on mRNA integrity of the two SARS-CoV-2 vaccines mRNA-1273 and BNT162b. The mRNA size of untreated and nebulized mRNA vaccines were analyzed by automated gel electrophoresis. The electropherograms of mRNA isolated from the non-nebulized samples (control condition) reveal a dominant peak between 3,500-4,000 for both vaccines (FIG. 10a,c), which is in line with the reported lengths of these mRNAs. To demonstrate the effect of nebulization on the mRNA integrity, electropherograms are shown in FIG. 10 for the BNT162b (cf. FIGS. 10a, 10b and 10c) and mRNA-1273 (cf. FIGS. 10d, 10e and 10f) vaccines before and after nebulization with vibrating mesh type 1 (VM1; blue), colliding jet (CJ; yellow), vibrating mesh type 2 (VM2; red), and with a nanotech membrane according to the invention (NM; green), all compared to the stock vaccines with and without RNase.

The nebulized samples were treated with RNase to degrade all mRNAs not encapsulated by lipid nano particles prior to electrophoretic analysis. The horizontal axis shows the RNA fragment size based on a series of RNA fragments with known lengths (200, 500, 1,000, 2,000, 4,000 and 6,000 nucleotides). For clarity, electropherograms were manually adjusted on the horizontal axis to align the full-length mRNA-vaccine peaks to the profiles obtained from the untreated stock solutions. The amount of RNA is shown as normalized fluorescence units on the vertical axis.

Naked mRNA molecules are susceptible to both enzymatic (e.g. RNase) and non-enzymatic degradation (e.g. shear). To uncouple these two degradation mechanisms RNA fragment lengths were investigated with and without adding RNase before after the nebulization process. RNase treatment before nebulization seems not to affect full-length mRNA of BNT162b (FIG. 10a) but decreased full-length mRNA from mRNA-1273 vaccine (dotted lines FIG. 10d). Notably, nebulization by the vibrating mesh 1 (VM1) and colliding jet (CJ) methods caused a 60-90% degradation of the mRNA in the vaccine (P<0,001), as observed by the decrease of the full-length mRNA peak (FIG. 10b.10e). In contrast, only a minor decrease (10-25%, P<0,001) of full-length mRNA was observed after nebulization by the vibrating mesh 2 (VM2) and nanotech membrane (NM) method according to the invention.

RNase treatment appears to significantly decrease the full-length mRNA content of all nebulized samples, except the NM-nebulized samples (FIG. 10c,f). Strikingly, even though full-length mRNA seems not substantially affected by the VM2 method, the subsequent addition of RNase degraded the mRNA to a large extent, indicating that the lipid nano particles were ruptured or dysfunctional after nebulization by the VM2 method. The resulting formulation was degraded to an extent comparable to the results of the VM1 and CJ method after adding RNase (FIG. 10c,f). In contrast, after RNase treatment the nebulized samples obtained with the NM method showed considerably less mRNA reduction and remained comparable with the stock solutions, showing that there is hardly any degradation (BNT162b: 10.1±1.4 and 7.7±1.6 ng/μl; mRNA-1273: 33.8±2.6 and 26.4±2.7 ng/μl for stock and after NM-nebulization).

To determine what energy levels involved in the nebulization method cause reversible deformation of the lipid nano particles versus irreversible rupture, the fraction of mRNA remaining intact after nebulization as a function of the energy dissipation for all three nebulization methods of the two vaccine formulations with and without adding RNase are plotted in FIG. 11A and FIG. 11B, respectively.

For the vibrating mesh (VM) technology, two commercially available nebulizers were used: VM1, a Pari eflow (Pari GmbH) and VM2, an Aerogen Solo (Aeroneb) nebulizer. The reservoirs of VM1 and VM2 were prefilled with 2-4 ml of the sample containing vaccine. The generated aerosols were collected in 30 mm diameter glass tubes and the collection time was measured for obtaining ca. 0.5-1 ml of nebulized fluid. A substantial increase in throughput with time (e.g., from 0.3 ml/min to 0.8 ml/min) was found for both vibrating mesh nebulizers, which is probably due to a temperature increase of the formulation due to dissipated vibration energy in the reservoir.

For the colliding jet (CJ) method, a Respimat (Boehringer Ingelheim) was used. A plastic cartridge of a reusable Respimat nebulizer was prefilled with the vaccine formulation and 60 doses or shots of 15 μL each were collected in a glass tube. Each shot had a duration of about 2 seconds. Reloading the spring of the device also took about 2 seconds.

For the nanotech membrane (NM) method according to the present invention, nanotech membrane chips were made with silicon semiconductor technology. In a silicon body a free hanging silicon nitride membrane was made with a thickness of 800 nanometer and in this membrane 85 pores with a 1.9 nm diameter were made by reactive ion etching. A syringe or spray pump was first prefilled with 1 ml vaccine formulation, which was manually pressed through the nanotech membrane nozzle at a pressure of 20 bar with a flowrate of 2-3 ml/min, and collected in a 10 mm diameter glass tube. All experiments were performed for both mRNA-1273 and BNT162b vaccines and reproduced three times. mRNA isolation

To isolate pure vaccine mRNA, 300 μl Trizol LS reagent was added to the untreated samples. Phenol and guanidine isothiocyanate used in this Trizol LS method lyse the lipid nano particles and protect the released vaccine mRNA against degradation by endonucleases. Next, the sample/Trizol LS mixtures were processed according to the manufacturer's instructions until ±230 μl of vaccine mRNA-containing water phase was obtained. The vaccine mRNA was purified and concentrated from the water phase into 12 μl using the RNeasy MinElute Cleanup Kit (Qiagen) according to the procedure described in Appendix D of the product manual. This RNA-isolation procedure was also done for nebulized samples and nebulized+RNase treated samples.

mRNA Quantification

Automated gel electrophoresis of the purified vaccine mRNA from all samples was performed on an Agilent 2200 TapeStation system using Agilent RNA ScreenTapes (Agilent). Electrophoresis profiles were rendered and quantified by the software Agilent 15 Tape Station System Software (Rev. 4.1.1). The concentration of the full-length vaccine-mRNA moiety peak was quantified by manually defining regions tightly around the full-length vaccine-mRNA peak, preventing the inclusion of degraded vaccine mRNA as much as possible. The RNA concentration within the defined regions was calculated by the software and used as a measure for the concentration of full-length vaccine mRNA in the samples.

### RNase Treatment

The freshly nebulized vaccine samples and stock vaccine samples (control condition) were split into two equal volumes of 100 μl. One subsample was treated for 5 minutes at 37° C. with RNase Cocktail Enzyme Mix (Thermo Fisher Scientific), containing a combination of RNaseA and RNaseT1 at a final concentration of 5 and 200 U/ml, respectively. This RNase treatment will completely degrade any vaccine mRNA that is not protected by intact lipid nano particle. After the RNase treatment, 300 μl Trizol LS reagent (Thermo Fisher Scientific) was added to the mixture to inactivate the RNases.

### Nebulization Energy Input

For the colliding jet nebulizer the spring constant of the spring to load the device was measured and from this, the energy stored in the spring and subsequently put into the sprayed formulation (15 μl) was directly calculated by using an energy transfer efficiency of 40-60%. For the nanotech membrane nebulizer, the amount of energy added to the spray was calculated from the pressure exerted on the piston of the syringe and the volume change.

For the vibrating mesh nebulizer, the formulation was sprayed for 60 sec and the temperature increase of the reservoir fluid was measured. This was done for three different amounts of reservoir fluid (1 g, 3 g and 6 g). The temperature increase was multiplied by the specific heat of water and the amount of liquid in the reservoir to arrive at the amount of energy taken up by the reservoir fluid during nebulization. The energy taken up by the reservoir fluid was extrapolated to a reservoir fluid amount of 0 g to get an indication of the amount of energy added to the sprayed fluid. This amount was divided by the amount of liquid that was sprayed to arrive at the energy density in J/g added to the sprayed fluid.

FIG. 11A depicts the fraction of intact mRNA without adding RNase. The absence of RNase means that lipid nano particle rupture and subsequent breakage of the mRNA chains has solely occurred due to fluid mechanical shear forces. At an energy dissipation of about 10-20 J/g a certain transition from intact to non-intact mRNA can be seen. FIG. 11B depicts the fraction of intact mRNA with added RNase after nebulization. This implies that lipid nano particle degradation is due to both fluid mechanical forces and enzymatic RNase activity.

A transition from intact to non-intact can be seen at an energy dissipation of about 5-10 J/g. Combining these figures, a picture emerges that lipid nano particles start to rupture at an energy dissipation of about 5-10 J/g, releasing mRNA chains, and that fluid-mechanical breakage of these (naked) mRNA chains happens at energy densities exceeding 10-20 J/g. The process of nebulizing a liquid formulation containing fragile long molecules, hence, according to the invention is preferably performed such that less than 20 Joule/g, and preferably less than 10 J/gram, of energy is dissipated in the liquid to be nebulized.

FIG. 12 shows a particular embodiment of a nasal adapter 77 for use with nasal spray device according to the invention. The nasal adapter comprises the spray nozzle 70 of the nasal spray device and has moreover means for generating two additional air streams 71,73. These means enable a co-axial air pit flow 71 with a high velocity along the path of the liquid micro-jets 72 that are released by the spray nozzle 70. This co-axial pit flow will prevent coalescence of droplets within the jets that are released by the spray nozzle chip 70. With preference the velocity of the pit air flow 71 is initially higher than the initial velocity of these microjets 72.

Additionally, said means allow a sheet flow of air 73 to be generated along the interior wall off adapter 77, indicated with the white dotted arrows, as the user inhales. This sheet flow 73 counteracts turbulence along the interior wall of airstreams containing microjet droplets that would otherwise hit the wall of the nasal adapter interface. The housing of the nasal adapter 77 has a conical shaped top that is intended to lie substantially sealingly within one of the nostrils of the user while directing the microjet droplets into the nasal cavity user. The additional air streams 71,73 may be generated passively as the user inhales through the nostril or they may be generated actively by an auxiliary supply of pressurized air, using appropriate means like a ventilator or a pressurized gas that is released once the user inhales.

All in all the depicted nasal spray device according to the present invention provides an intranasal spray atomiser that maintains the convenience and simplicity of a swirl nozzle device, while having several benefits compared with to a traditional swirl nozzle, like:
a low dosing volume required for nebulisation, than may be as low as 25 μL while still providing an adequate therapeutic volume that is being administered;
a droplet size that is independent of the liquid flow rate and independent of the liquid formulation; allowing a slowly moving soft mist of ultrafine droplets;
a narrow droplet size distribution merely dependent on the diameter of the micropores that allows tailoring the deposition to specifically the nasal regions to be targeted and avoiding deep respiratory deposition;
an optimal deposition efficacy in nasal cavity due to low plume velocity below 1.0 m/s and narrow droplet size distribution;
an optimised plume cone angle between 10 and 25 degrees to allow wider deposition in the nasal cavity;
limited dripping and clearance of liquid due to larger surface coverage of deposition
low impact force due to reduced plume velocity and smaller droplet size
Luer-lock fitting capability for easy attachment to e.g. existing medical Luer-lock syringes
parallel inspiration flow of ambient air along the device through purposely designed air slits.

While various embodiments of the invention have been described above, it should be understood that they have been presented by way of example, and not limitation. It is apparent to persons skilled in the relevant arts that various changes in form and detail can be made therein. Thus, the invention should not be limited by any of the above-described embodiments, but should be defined only in accordance with the following claims and their equivalents.

Particularly the nasal spray device may comprises a silicon chip serving as nozzle body carrying the nozzle membrane, for instance a nozzle membrane formed from a silicon nitride ceramic layer with a thickness of the order of one micron. Micropores (orifices) may be etched through such layer on a nanometre to micrometre scale using ultra high precision photo lithographic imaging, masking and etching techniques as available in modern semiconductor manufacturing technology.

The size of the micropores inside the spray chip determines predominantly the initial size of the droplets that are released in the spray. This in turn determines to a large extent the target area where the droplets will mainly land in the respiratory system. Micropores with a size between 3 micron and 8 micron will deliver droplets that are typically larger than 10 micrometer and, as a consequence, mainly target the nasal cavity, particularly the nasal turbinates, without penetrating deeper into the respiratory system, as these droplets larger than 10 micron will generally not be able to pass the nasopharynx and will remain instead in the upper airways. Droplets of a size between 1 micron and 10 micron, on the other hand, may generally transfer through the nasopharynx to eventually deposit predominantly in the lower airways (trachea and alveoli). Droplets smaller than 1 micron will not be deposited at all, but will be exhaled substantially completely to be received by the deeper portion of the respiratory system, notably the bronchi and the lungs.

As shown in FIG. 5B, the nasal spray device according to the invention enables an internasal delivery with a high coverage on the internasal tissues. For many prophylactic and other applications such internasal delivery with a high coverage will be sufficient. Some other prophylactic and other conditions, however, (also) require a deeper deposition in the lower airways. A particular embodiment of the nasal spray device according to the invention is therefore configured to deliver a liquid formulation to both upper and lower airways, in a single inhalation step when inhaled through the nose.

To that end, the same or a further microchip within the nasal spray device may concurrently be fed with the pressurized liquid, comprising a second group of micropores having a diameter below 3 micron, for instance between 1.5 and 3 micron. The droplets emanating from these micropores tend to have a size below 10 micron that typically will not be captured in the nasal cavity but instead will pass the nose to enter the downstream respiratory tract, when inhaled.

The larger droplets from the first group of micropores, between 3 and 8 micron in diameter, are intended to be captured in the upper airways, particularly in the nasal vestibule, nasal turbinate and olfactory region. The second group of micropores with an orifice smaller than 3 micron, will create droplets that are sufficiently small to be able to target nasopharynx and lower airways (lungs) when inhaled through the nose. Due to the smaller size of these droplets and, hence, their lower mass, the smaller droplets have a lower kinetic energy and a low plume velocity. These smaller droplets follow the airstream through the upper airways into the lower airways. By varying the ratio between the number of small and large pores it is possible to tailor the ratio of prophylactic formulation deposition between the upper and lower airways.

Such nasal device according to the invention, hence, may deliver a liquid formulation containing for instance a prophylactic, pharmaceutical drug, also to both the upper and lower airways, in a single inhalation step when inhaled through the nose. The size of the micropores inside the spray nozzle predominantly determines the initial droplet size and thereby controls the target location of deposition in the upper and/or lower airways. When the micropores are combined having a pore size between 3 micron and 8 micron in one group and having a pore size smaller than 3 micron in another group, the device generate a nasal spray that will target the entire respiratory tract, when inhaled. A unique dual therapy is thus offered by a single nasal spray device for use with conditions that require coverage of both the nasal mucous membrane as well as that of the deeper respiratory region, particularly the bronchi and lungs.

1. A nasal spray device, comprising a nostril cap having an end portion to be received inside a nostril cavity of a user, wherein said nostril cap comprises a spray cavity that opens through said end portion, and wherein said nostril cap comprises a spray nozzle that is configured and suspended to receive a liquid formulation at a inlet and to release a liquid spray of said liquid formulation at an outlet that opens into said spray cavity, wherein said spray nozzle comprises a membrane between said inlet and said outlet having a plurality of micropores of a substantially identical size of between 1 micron and 10 micron, particularly between 3 micron and 8 micron, that carry said liquid formulation to be discharged as a spray jet from the device, wherein said micropores are configured to release a jet of microdroplets under an angle of deflection with respect to a axial centreline of said respective micropore and wherein said micropores are distributed to form together a substantially conically shaped diverging spray plume having an apex.

2. The nasal spray device according to claim 1, wherein said conically shaped spray plume has an apex of around 5 degrees.

3. The nasal spray device according to claim 1, wherein said conically shaped spray plume has an apex of between 5 and 30 degrees, particularly of between 20 and 25 degrees, and more particularly of around 20 degrees.

4. The nasal spray device according to claim 1, wherein a chamber is provided for containing a predetermined dose of said liquid formulation, particularly having a volume of between 25 and 150 microlitre, wherein a manually energizable positive displacement pump is configured to pressurize said dose of said liquid formulation and forcing the liquid formulation under an elevated operating pressure of between 5 and 15 bar to said inlet of said spray nozzle that releases said predetermined dose of said liquid formulation.

5. The nasal spray device according to claim 4, wherein said pump comprises a manually actuated piston that pressurizes said dose of said liquid formulation to said operating pressure in a single stroke of said piston.

6. The nasal spray device according to claim 4, wherein said pump comprises a multi shot nasal pump that is provided with a dosing chamber for holding said dose of said liquid formulation at least temporarily and releasing said dose to said spray nozzle under said operating pressure.

7. The nasal spray device according to claim 4, wherein said pump is formed by a medical syringe.

8. The nasal spray device according to claim 4, wherein said pump opens into a male Luer tip, in wherein said nostril cap is receivable onto said male Luer tip having an inlet that provides a female Luer slip connection with said male Luer tip and having an outlet to release said nasal spray, and wherein said nostril cap comprises said spray nozzle between said inlet and said outlet.

9. The nasal spray device according to claim 1, wherein said nostril-cap comprises at least one inhalation channel that carries a parallel airflow of ambient air along with said spray towards the outlet, while the user inhales.

10. The nasal spray device according to claim 1, wherein said nostril-cap tapers down towards said outlet to have a close nasal fit.

11. The nasal spray device according to claim 1, wherein said membrane comprises a ceramic layer, wherein said micropores extend over a thickness of said ceramic layer.

12. The nasal spray device according to claim 11, wherein said ceramic layer is a silicon nitride layer lying on a carrier body of a semiconductor material, particularly of silicon, wherein said carrier body is provided with at least one cavity underneath said nitride layer, wherein said at least one cavity opens downstream into at least one micropore of said plurality of micropores, and wherein said at least one cavity is connected upstream to an outlet of said pump.

13. The nasal spray device according to claim 1, wherein the aerosol spray emanates from the micropores of the membrane in the spray nozzle in Rayleigh jets that subsequently break up into particles of the aerosol spray.

14. The nasal spray device according to claim 1, wherein the reservoir contains a pharmaceutically active liquid formulation, particularly one containing nanoparticles, such as formulations containing complex proteins, peptides, long chain DNA & RNA, large vesicles, liposomes, lipid nanoparticles, mRNA vaccines, mRNA lipid nanoparticles and antibodies.

15. The nasal spray device according to claim 1, wherein said membrane of said spray nozzle comprises a second group of micropores of substantially identical size below 3 micron through which the liquid formulation passes as the liquid formulation is discharged from the device.

16. The nasal spray device according to claim 1, wherein said liquid formulation comprises nano-particles of a size δ, wherein said nano-particles have a maximum size δₘₐₓ before rupture upon elongation and wherein said micropores have a length L that is smaller than the pore diameter D times δₘₐₓ/δ (L<D·δₘₐₓ/δ), in particular L<4D.

17. The nasal spray device according to claim 14, wherein the liquid formulation comprises macromolecules with a chain length λ, and wherein said micropores have a length L that is smaller than the chain length λ of the macromolecules, in particular L is smaller than 1 micron.

18. A method of nebulizing a liquid formulation, particularly a liquid formulation containing macro molecules, more particularly containing biological molecules, wherein said liquid formulation is subjected to a treatment to convert said liquid formulation into a mist of said liquid formulation, and wherein said liquid formulation is subjected to an energy not exceeding 20 Joule/gram, preferably not exceeding 10 Joule/gram, while converting said liquid formulation into a mist of said liquid formulation, particularly forming a soft mist spray plume.

19. A method according to claim 18, wherein said liquid formulation is pressurized to an operating pressure to create at least a pressurized dose of said liquid formulation, and wherein said pressurized dose of said liquid formulation is forced through at least one micropore, particularly trough a plurality of micropores, in a membrane of a Rayleigh break-up type spray nozzle.

20. Method according to claim 19, wherein said liquid formulation is pressurized to an operating pressure of between 5 and 15 bar using a pump, particularly a manually energizable pump to create a pressurized dose of said liquid formulation, and wherein said pressurized dose of said liquid formulation is forced through a group of micropores in said membrane of a Rayleigh type spray nozzle, said micropores having a size smaller than 8 micron, to cause said liquid formulation to breakup in at least one spray jet of liquid droplets having a substantially identical initial droplet size, causing said mist to propagate with a spray plume propagation velocity of less than 1 m/s.

21. The method according to claim 19, wherein said liquid formulation contains nano-particles of a size λ and said liquid droplets contain at least one nano-particle of said nano-particles, wherein said nano-particles have a maximum size λₘₐₓ before breakage upon elongation; and wherein said liquid formulation is subjected to a shear rate γ [per second] while passing through a micropore; and wherein said liquid formulation is exposed within said micropore to said shear rate γ during a shear time Δt that is less than λₘₐₓ/(λ·γ) seconds.

22. The method according to claim 19, wherein said liquid formulation comprises nano-particles, wherein said nano-particles have a size δ and a maximum size δₘₐₓ before rupture upon elongation and wherein said micropores have a length L that is smaller than D·δₘₐₓ/δ, in particular L<4.D, wherein D is a diameter of the micropores.

23. The method according to claim 19, wherein said liquid formulation comprises macromolecules with a chain length λ, and wherein said micropores have a length L that is smaller than the chain length λ of the macromolecules, in particular L is smaller than 1 micron.

24. The method according to claim 18, wherein said liquid formulation comprises nano-particles taken from a group, containing complex proteins, large biological molecules, long chain DNA & RNA, large vesicles, liposomes, bacteriophages, lipid nanoparticles, mRNA vaccines, mRNA lipid nanoparticles and antibodies.
